(19) United States
(12) Patent Application Publication
(10) Pub. No.: US 2016/0374643 A1
(43) Pub. Date: Dec. 29, 2016

(54) METHOD AND SYSTEM FOR ENHANCED VISUALIZATION BY AUTOMATICALLY ADJUSTING ULTRASOUND NEEDLE RECOGNITION PARAMETERS

(71) Applicant: GENERAL ELECTRIC COMPANY, Schenectady, NY (US)

(72) Inventors: Menachem Halmann, Wauwatosa, WI (US); Feng Lin, WuXi (CN); Jeffrey Scott Peiffer, Wauwatosa, WI (US); Eunju Kang, Wauwatosa, WI (US); Bo Li, WuXi (CN); David J. Bates, Wauwatosa, WI (US)

(73) Assignee: GENERAL ELECTRIC COMPANY, Schenectady, NY (US)

(21) Appl. No.: 15/039,710

(22) PCT Filed: Dec. 31, 2013
(86) PCT No.: PCT/CN2013/091021

§ 371 Date: May 26, 2016

(51) Int. Cl. A61B 8/08; G01S 7/52; G01S 15/89; A61B 8/00

(52) U.S. Cl. CPC A61B 8/0841; A61B 8/5207; A61B 8/4483; G01S 7/5208; G01S 15/899

(57) ABSTRACT

An ultrasound device determines a position and orientation of a surgical instrument based at least in part on tracking information, such as magnetic field strength, emitted by an emitter of a tracking system and detected by a sensor of the tracking system. The sensor and the emitter are attached to or within a different one of a probe of the ultrasound device and the surgical instrument. The ultrasound device determines an ultrasound imaging parameter, such as an ultrasound beam steering angle, based at least in part on the determined position and orientation of the surgical instrument. The ultrasound device applies the ultrasound imaging parameter to acquire ultrasound image data of a target. The ultrasound device generates an ultrasound image based on the acquired ultrasound image data of the target. The ultrasound image includes a representation of the surgical instrument. The surgical instrument may be a needle.

### FIELD OF THE INVENTION

Certain embodiments of the invention relate to ultrasound imaging. More specifically, certain embodiments of the invention relate to a method and system for enhanced visualization of a surgical needle in ultrasound data by automatically adjusting ultrasound needle recognition parameters.

### BACKGROUND OF THE INVENTION

Ultrasound imaging is a medical imaging technique for imaging organs and soft tissues in a human body. Ultrasound imaging uses real time, non-invasive high frequency sound waves to produce a two-dimensional (2D) image and/or a three-dimensional (3D) image.

In conventional ultrasound imaging, an operator of an ultrasound system can acquire images in various modes by, for example, manually activating a button to toggle between the modes. For example, an operator can toggle between a non-compounding mode and compounding modes that may include electronically steering left or right (in 2D), or left, right, in, or out (in 3D). The term “compounding” generally refers to non-coherently combining multiple data sets to create a new single data set. The plurality of data sets may each be obtained from imaging the object from different angles, using different imaging properties, such as, for example, aperture and/or frequency, and/or imaging nearby objects (such as slightly out of the plane steering). These compounding techniques may be used independently or in combination to improve image quality.

Ultrasound imaging may be useful in positioning an instrument at a desired location inside a human body. For example, in order to perform a biopsy on a tissue sample, it is important to accurately position a biopsy needle so that the tip of the biopsy needle penetrates the tissue to be sampled. By viewing the biopsy needle using an ultrasound imaging system, the biopsy needle can be directed toward the target tissue and inserted to the required depth. Thus, by visualizing both the tissue to be sampled and the penetrating instrument, accurate placement of the instrument relative to the tissue can be achieved.

A needle is a specular reflector, meaning that it behaves like a mirror with regard to the ultrasound waves reflected off of it. The ultrasound is reflected away from the needle at an angle equal to the angle between the transmitted ultrasound beam and the needle. Ideally, an incident ultrasound beam would be substantially perpendicular with respect to a surgical needle in order to visualize the needle most effectively. The smaller the angle at which the needle is inserted relative to the axis of the transducer array, i.e., the imaginary line normal to the face of the transducer array, the more difficult it becomes to visualize the needle. In a typical biopsy procedure using a linear probe, the geometry is such that most of the transmitted ultrasound energy is reflected away from the transducer array face and thus is poorly detected by the ultrasound imaging system.

In some cases, an operator of a conventional ultrasound imaging system can improve visualization of a surgical needle by toggling a steer button such that an angle at which a transmitted ultrasound beam impinges upon the needle is increased, which increases the system's sensitivity to the needle because the reflection from the needle is directed closer to the transducer array. A composite image of the needle can be made by acquiring a frame using a linear transducer array operated to scan without steering (i.e., with beams directed normal to the array) and one or more frames acquired by causing the linear transducer array to scan with beams steered toward the needle. The component frames are combined into a compound image by summation, averaging, peak detection, or other combinational means. The compounded image may display enhanced specular reflector delineation than a non-compounded ultrasound image, which serves to emphasize structural information in the image. However, an operator of a conventional ultrasound imaging system may find it difficult and/or inconvenient to manually toggle a steer button to provide the electronic steering. For example, an operator holding an ultrasound probe in one hand and a surgical needle in the other hand may have to put down and/or remove the needle from a patient in order to provide the manual steering adjustments.

Further limitations and disadvantages of conventional and traditional approaches will become apparent to one of skill in the art, through comparison of such systems with some aspects of the present invention as set forth in the remainder of the present application with reference to the drawings.

### BRIEF SUMMARY OF THE INVENTION

A system and/or method is provided for enhanced visualization of a surgical needle in ultrasound data by automatically adjusting ultrasound needle recognition parameters, substantially as shown in and/or described in connection with at least one of the figures, as set forth more completely in the claims.

These and other advantages, aspects and novel features of the present invention, as well as details of an illustrated embodiment thereof, will be more fully understood from the following description and drawings.

### BRIEF DESCRIPTION OF SEVERAL VIEWS OF THE DRAWINGS

FIG. 1 is a block diagram of an exemplary ultrasound system that is operable to provide enhanced visualization of a surgical needle in ultrasound data by automatically adjusting ultrasound needle recognition parameters, in accordance with an embodiment of the invention.

FIG. 2 is a flow chart illustrating exemplary steps that may be utilized for providing enhanced visualization of a surgical needle in ultrasound data by automatically adjusting ultrasound needle recognition parameters, in accordance with an embodiment of the invention.

### DETAILED DESCRIPTION OF THE INVENTION

Certain embodiments of the invention may be found in a method and system for enhanced visualization of a surgical needle in ultrasound data by automatically adjusting ultrasound needle recognition parameters.

The foregoing summary, as well as the following detailed description of certain embodiments will be better understood when read in conjunction with the appended drawings. To the extent that the figures illustrate diagrams of the functional blocks of various embodiments, the functional blocks are not necessarily indicative of the division between hardware circuitry. Thus, for example, one or more of the functional blocks (e.g., processors or memories) may be implemented in a single piece of hardware (e.g., a general purpose signal processor or a block of random access memory, hard disk, or the like) or multiple pieces of hardware. Similarly, the programs may be stand alone programs, may be incorporated as subroutines in an operating system, may be functions in an installed software package, and the like. It should be understood that the various embodiments are not limited to the arrangements and instrumentality shown in the drawings. It should also be understood that the embodiments may be combined, or that other embodiments may be utilized and that structural, logical and electrical changes may be made without departing from the scope of the various embodiments of the present invention. The following detailed description is, therefore, not to be taken in a limiting sense, and the scope of the present invention is defined by the appended claims and their equivalents.

As used herein, an element or step recited in the singular and proceeded with the word “a” or “an” should be understood as not excluding plural of said elements or steps, unless such exclusion is explicitly stated. Furthermore, references to “one embodiment” are not intended to be interpreted as excluding the existence of additional embodiments that also incorporate the recited features. Moreover, unless explicitly stated to the contrary, embodiments “comprising” or “having” an element or a plurality of elements having a particular property may include additional elements not having that property.

Also as used herein, the term “image” broadly refers to both viewable images and data representing a viewable image. However, many embodiments generate (or are configured to generate) at least one viewable image. In addition, as used herein, the phrase “image” is used to refer to an ultrasound mode such as B-mode, CF-mode and/or sub-modes of CF such as TVI, Angio, B-flow, BMI, BMI Angio, and in some cases also MM, CM, PW, TVD, CW where the “image” and/or “plane” includes a single beam or multiple beams.

Furthermore, the term processor or processing unit, as used herein, refers to any type of processing unit that can carry out the required calculations needed for the invention, such as single or multi-core: CPU, Graphics Board, DSP, FPGA, ASIC or a combination thereof.

It should be noted that various embodiments described herein that generate or form images may include processing for forming images that in some embodiments includes beamforming and in other embodiments does not include beamforming. For example, an image can be formed without beamforming, such as by multiplying the matrix of demodulated data by a matrix of coefficients so that the product is the image, and wherein the process does not form any “beams”. Also, forming of images may be performed using channel combinations that may originate from more than one transmit event (e.g., synthetic aperture techniques).

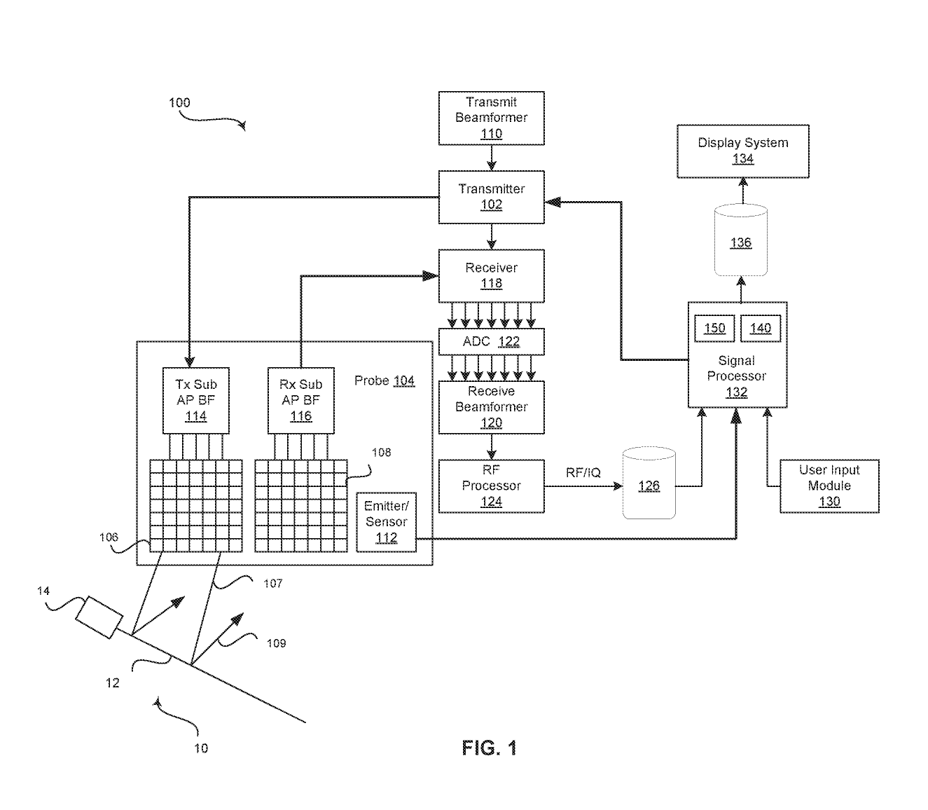

In various embodiments, ultrasound processing to form images is performed, for example, including ultrasound beamforming, such as receive beamforming, in software, firmware, hardware, or a combination thereof. One implementation of an ultrasound system having a software beamformer architecture formed in accordance with various embodiments is illustrated in FIG. 1.

FIG. 1 is a block diagram of an exemplary ultrasound system 100 that is operable to provide enhanced visualization of a surgical needle 10 in ultrasound data by automatically adjusting ultrasound needle recognition parameters, in accordance with an embodiment of the invention. Referring to FIG. 1, there is shown a surgical needle 10 and an ultrasound system 100. The surgical needle 10 comprises a needle portion 12 and a needle emitter/sensor 14. The ultrasound system 100 comprises a transmitter 102, an ultrasound probe 104, a transmit beamformer 110, a receiver 118, a receive beamformer 120, a RF processor 124, a RF/IQ buffer 126, a user input module 130 a signal processor 132, an image buffer 136, and a display system 134.

The surgical needle 10 comprises a needle portion 12 that includes a distal insertion end and a proximal hub end. A needle emitter/sensor 14 is attached to the needle portion 12 at the proximal hub end and/or is secured within a housing attached to the proximal hub end of the needle portion 12. The needle emitter/sensor 14 can be, for example, an emitter or sensor that corresponds with a sensor or emitter of the probe emitter/sensor 112 of the ultrasound system 100 probe 104. The emitter may be a permanent magnet that corresponds with a sensor, an electromagnetic coil that corresponds with a receiver, an optical source that corresponds with a photo-detector, or any suitable emitter that corresponds with a sensor to form a tracking system. As an example, the needle emitter 14 may comprise a magnetic element that generates a magnetic field detectable by one or more sensors of the probe sensor 112 to enable the position and orientation of the surgical needle 10 to be tracked by the ultrasound system 100.

The transmitter 102 may comprise suitable logic, circuitry, interfaces and/or code that may be operable to drive an ultrasound probe 104.

The ultrasound probe 104 may comprise suitable logic, circuitry, interfaces and/or code, which may be operable to perform some degree of beam steering, which may be perpendicular to the scan plane direction. The ultrasound probe 104 may comprise a two dimensional (2D) or three dimensional (3D) array. In an exemplary embodiment of the invention, the ultrasound probe 104 may comprise a three dimensional (3D) array of elements that is operable to steer a beam in the desired spatial 3D direction. The ultrasound probe 104 may comprise a group of transmit transducer elements 106 and a group of receive transducer elements 108, that normally constitute the same elements. The ultrasound probe 104 may comprise an emitter/sensor 112 for coordinating with a needle emitter/sensor 14 to track the position of a surgical needle 10. The emitter may be a permanent magnet that corresponds with a sensor, an electromagnetic coil that corresponds with a receiver, an optical source that corresponds with a photo-detector, or any suitable emitter that corresponds with a sensor to form a tracking system. As an example, the needle emitter 14 may comprise a magnetic element that generates a magnetic field detectable by one or more sensors of the probe sensor 112 to enable the position and orientation of the surgical needle 10 to be tracked by the ultrasound system 100.

The transmit beamformer 110 may comprise suitable logic, circuitry, interfaces and/or code that may be operable to control the transmitter 102 which, through a transmit sub-aperture beamformer 114, drives the group of transmit transducer elements 106 to emit ultrasonic transmit signals 107 into a region of interest (e.g., human, animal, underground cavity, physical structure and the like). The transmitted ultrasonic signals 107 may be back-scattered from structures in the object of interest, like blood cells, and surgical instruments in the object of interest, like a surgical needle 10, to produce echoes 109. The echoes 109 are received by the receive transducer elements 108.

The group of receive transducer elements 108 in the ultrasound probe 104 may be operable to convert the received echoes into analog signals, undergo sub-aperture beamforming by a receive sub-aperture beamformer 116 and are then communicated to a receiver 118.

The receiver 118 may comprise suitable logic, circuitry, interfaces and/or code that may be operable to receive and demodulate the signals from the receive sub-aperture beamformer 116. The demodulated analog signals may be communicated to one or more of the plurality of A/D converters 122.

The plurality of A/D converters 122 may comprise suitable logic, circuitry, interfaces and/or code that may be operable to convert the demodulated analog signals from the receiver 118 to corresponding digital signals. The plurality of A/D converters 122 are disposed between the receiver 118 and the receive beamformer 120. Notwithstanding, the invention is not limited in this regard. Accordingly, in some embodiments of the invention, the plurality of A/D converters 122 may be integrated within the receiver 118.

The receive beamformer 120 may comprise suitable logic, circuitry, interfaces and/or code that may be operable to perform digital beamforming processing on the signals received from the plurality of A/D converters 122. The resulting processed information may be converted back to corresponding RF signals. The corresponding output RF signals that are output from the receive beamformer 120 may be communicated to the RF processor 124. In accordance with some embodiments of the invention, the receiver 118, the plurality of A/D converters 122, and the beamformer 120 may be integrated into a single beamformer, which may be digital.

The RF processor 124 may comprise suitable logic, circuitry, interfaces and/or code that may be operable to demodulate the RF signals. In accordance with an embodiment of the invention, the RF processor 124 may comprise a complex demodulator (not shown) that is operable to demodulate the RF signals to form I/Q data pairs that are representative of the corresponding echo signals. The RF or I/Q signal data may then be communicated to an RF/IQ buffer 126.

The RF/IQ buffer 126 may comprise suitable logic, circuitry, interfaces and/or code that may be operable to provide temporary storage of the RF or I/Q signal data, which is generated by the RF processor 124.

The user input module 130 may be utilized to input patient data, surgical instrument data, scan parameters, settings, configuration parameters, change scan mode, and the like. In an exemplary embodiment of the invention, the user input module 130 may be operable to configure, manage and/or control operation of one or more components and/or modules in the ultrasound system 100. In this regard, the user input module 130 may be operable to configure, manage and/or control operation of transmitter 102, the ultrasound probe 104, the transmit beamformer 110, the receiver 118, the receive beamformer 120, the RF processor 124, the RF/IQ buffer 126, the user input module 130, the signal processor 132, the image buffer 136, and/or the display system 134

The signal processor 132 may comprise suitable logic, circuitry, interfaces and/or code that may be operable to process acquired tracking information (i.e., magnetic field strength data or any suitable tracking information from sensor 112 or 14) for computing adjusted ultrasound needle recognition parameters, and process ultrasound information (i.e., RF signal data or IQ data pairs) for presentation on a display system 134. The signal processor 132 is operable to perform one or more processing operations to determine the position and orientation information of a surgical needle 10. The signal processor 132 is operable to perform one or more processing operations according to a plurality of selectable ultrasound modalities on the acquired ultrasound information. In an exemplary embodiment of the invention, the signal processor 132 may be operable to perform compounding, motion tracking, and/or speckle tracking. Acquired ultrasound information may be processed in real-time during a scanning session as the echo signals are received. Additionally or alternatively, the ultrasound information may be stored temporarily in the RF/IQ buffer 126 during a scanning session and processed in less than real-time in a live or off-line operation. In the exemplary embodiment, the signal processor 132 may comprise a spatial compounding module 140 and a processing module 150.

The ultrasound system 100 may be operable to continuously acquire ultrasound information at a frame rate that is suitable for the imaging situation in question. Typical frame rates range from 20-70 but may be lower or higher. The acquired ultrasound information may be displayed on the display system 134 at a display-rate that can be the same as the frame rate, or slower or faster. An image buffer 136 is included for storing processed frames of acquired ultrasound information that are not scheduled to be displayed immediately. Preferably, the image buffer 136 is of sufficient capacity to store at least several seconds worth of frames of ultrasound information. The frames of ultrasound information are stored in a manner to facilitate retrieval thereof according to its order or time of acquisition. The image buffer 136 may be embodied as any known data storage medium.

The spatial compounding module 140 is optional and may comprise suitable logic, circuitry, interfaces and/or code that may be operable to combine a plurality of steering frames corresponding to a plurality of different angles to produce a compound image.

The processing module 150 may comprise suitable logic, circuitry, interfaces and/or code that may be operable to handle processing of tracking data to provide enhanced visualization of a surgical needle in ultrasound data by automatically adjusting ultrasound needle recognition parameters. In this regard, the processing module 150 may comprise suitable logic, circuitry, interfaces and/or code that may be operable to handle processing the acquired tracking information (i.e., magnetic field strength data or any suitable tracking information from sensor 112 or 14) for computing adjusted ultrasound needle recognition parameters. The signal processor 132 is operable to perform one or more processing operations to determine the position and orientation information of a surgical needle 10.

In an exemplary embodiment of the invention, X, Y, and Z coordinate positions of a needle emitter 14 with respect to the probe sensor(s) 112 can be determined in real-time by the signal processor 132 using tracking data, such as magnetic field strength data sensed by the probe sensor(s) 112. The position and orientation information determined by the signal processor 132, together with the length of the needle portion 12 and position of the needle emitter 14 with respect to the distal insertion end as known by or input into the signal processor 132, enable the signal processor 132 to accurately determine the position and orientation of the entire length of the surgical needle 10 with respect to the probe sensor(s) 112 in real-time. Because the signal processor 132 is able to determine the position and orientation of the needle 10 with respect to the probe sensor(s) 112, the position and orientation of the needle 10 with respect to an ultrasound image can also be accurately determined by the signal processor 132. The probe sensor(s) 112 are configured to continuously detect tracking data from the emitter 14 of the needle 10 during operation of the ultrasound system 100. This enables the signal processor 132 to continuously update the position and orientation of the needle 10 for use in automatically computing ultrasound needle recognition parameters. The ultrasound needle recognition parameters can include, for example, an ultrasound beam steering angle, gain, frequency, focal zone, transmit sub-aperture, receive sub-aperture, etc.

The ultrasound needle recognition parameters can be provided by the processing module 150 of the signal processor 132 to the transmitter 102 and/or transmit beamformer 110 to provide the conditions for emitting the ultrasonic transmit signals 107 into a region of interest, for example. As an example, the processing module 150 may be operable to control the steering of the ultrasound signals generated by the plurality of transmit transducer elements 106 and/or the plurality of receive transducer elements 108 to a plurality of angles.

In operation and in an exemplary embodiment of the invention, the probe 104 is placed against the patient skin, transmits an ultrasound beam 107 to a target within a patient, and receives ultrasound echoes 109 used to generate an ultrasound image. The ultrasound image of the target can be depicted on the display 134 of the ultrasound system 100. The system 100 is configured to detect the position and orientation of the surgical needle 10. Particularly, one or more sensors 112 of the probe 104 is configured to detect a magnetic field of the magnetic emitter 14 included with the needle 10. The sensor(s) 112 are configured to spatially detect the magnetic emitter 14 in three dimensional space. As such, during operation of the ultrasound system 100, magnetic field strength data emitted by the magnetic emitter 14 and sensed by the one or more sensors 112 is communicated to a processing module 150 of a signal processor 132 that continuously computes the real-time position and/or orientation of the needle 10. The real-time position and/or orientation of the needle 10 is used to automatically compute ultrasound needle recognition parameters, such as an ultrasound beam steering angle, a gain, and a frequency, among other things. The ultrasound needle recognition parameters are applied by the processing module 150 of the signal processor 132 to the transmitter 102 and/or transmit beamformer 110 to acquire enhanced ultrasound image data of the target by controlling the emission of the ultrasonic transmit signals 107 into a region of interest. The elevation beam width of the ultrasound beams 107 transmitted by the probe 104 is constant. The signal processor 132 generates an ultrasound image that comprises a representation of the needle based on the acquired ultrasound image data of the target. The representation may be an image of the needle 10 when the needle 10 is in-plane of the ultrasound image data, for example. Additionally and/or alternatively, the representation can be a virtual representation of the needle 10 overlaid on the ultrasound image of the target when, for example, the needle 10 is out-of-plane of the ultrasound image data. In various embodiments, the ultrasound image can be generated by compounding the ultrasound image data of the target.

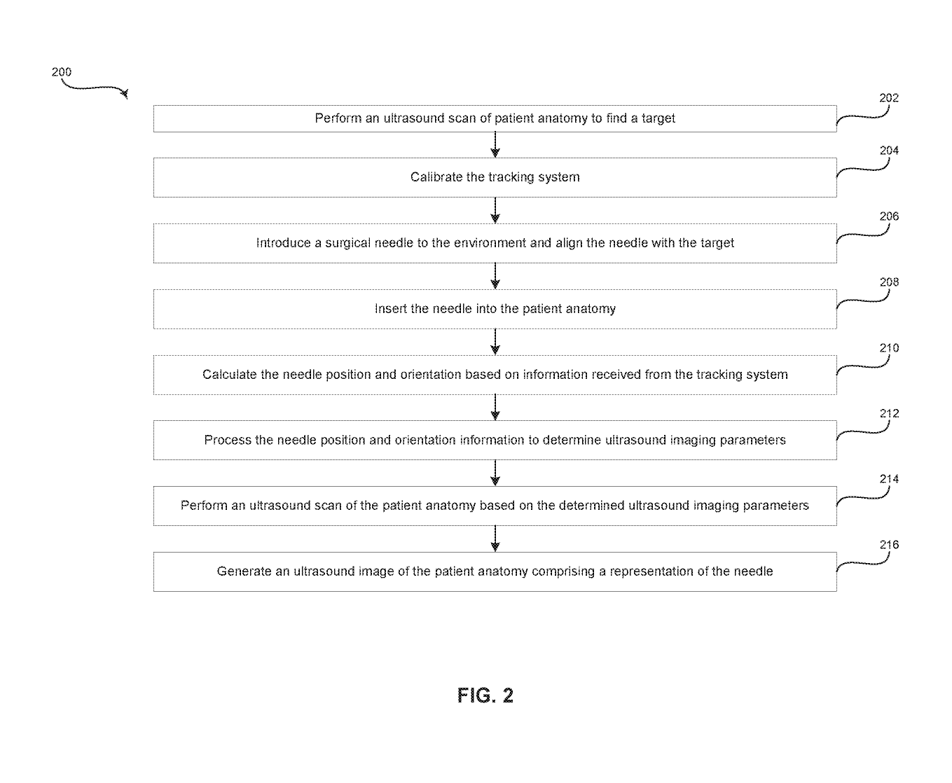

FIG. 2 is a flow chart illustrating exemplary steps that may be utilized for providing enhanced visualization of a surgical needle 10 in ultrasound data by automatically adjusting ultrasound needle recognition parameters, in accordance with an embodiment of the invention. Referring to FIG. 2, there is shown a flow chart 200 comprising exemplary steps 202 through 216. Certain embodiments of the present invention may omit one or more of the steps, and/or perform the steps in a different order than the order listed, and/or combine certain of the steps discussed below. For example, some steps may not be performed in certain embodiments of the present invention. As a further example, certain steps may be performed in a different temporal order, including simultaneously, than listed below.

In step 202, the ultrasound probe 104 in the ultrasound system 100 may be operable to perform an ultrasound scan of patient anatomy to find a target, such that the probe 104 is positioned at the target.

In step 204, a tracking system may be calibrated. For example, in a tracking system comprising a permanent magnet emitter 14 coupled to or within a surgical needle 10 and one or more sensors 112 coupled to or within a probe 104, the needle 10 may be removed from the surgical environment so that the tracking system can be calibrated to remove ambient magnetic fields detected by the sensor(s) 112.

In step 206, a surgical needle 10 can be introduced to the surgical environment and aligned with a target.

In step 208, the needle may be inserted into the patient anatomy.

In step 210, a processing module 150 of a signal processor 132 of the ultrasound system 100 can calculate a position and orientation of the needle 10 based at least in part on information received from the tracking system. For example, in a tracking system comprising a permanent magnet emitter 14 coupled to or within a surgical needle 10 and one or more sensors 112 coupled to or within a probe 104, the probe sensor(s) 112 can detect the magnet field change caused by the introduction of the permanent magnet emitter 14 of the needle 10 into the surgical environment. The probe sensor(s) 112 may provide the magnetic field strength data to the processing module 150 of the signal processor 132 such that X, Y, and Z coordinate positions of a needle emitter 14 with respect to the probe sensor(s) 112 can be determined in real-time. In particular, the position and orientation information determined by the processing module 150, together with the length of the needle portion 12 and position of the needle emitter 14 with respect to the distal insertion end as known by or input into the processing module 150, enable the processing module 150 to accurately determine the position and orientation of the entire length of the surgical needle 10 with respect to the probe sensor(s) 112 in real-time.

In step 212, the processing module 150 of the signal processor 132 can process the needle position and orientation information to automatically and dynamically determine ultrasound imaging parameters, such as ultrasound needle recognition parameters. The parameters may include, for example, ultrasound beam steering angle, gain, frequency, focal zone, transmit sub-aperture, and receive sub-aperture, among other things.

In step 214, the ultrasound probe 104 in the ultrasound system 100 may be operable to perform an ultrasound scan of patient anatomy based on the determined ultrasound imaging parameters. For example, the processing module 150 of the signal processor 132 can apply the ultrasound imaging parameters to the transmitter 102 and/or transmit beamformer 110 to acquire enhanced ultrasound image data of the target by controlling the emission of the ultrasonic transmit signals 107 into a region of interest. The elevation beam width of the ultrasonic transmit signals 107 transmitted by the probe 104 is constant.

In step 216, the signal processor 132 can generate an ultrasound image of the patient anatomy comprising a representation of the needle 10. For example, the representation may be an image of the needle 10 when the needle 10 is in-plane of the ultrasound image data. As another example, the representation can be a virtual representation of the needle 10 overlaid on the ultrasound image of the target when the needle is out-of-plane of the ultrasound image data. In various embodiments, spatial compounding module 140 can generate the ultrasound image by compounding the ultrasound image data of the target.

Aspects of the present invention provide a method 200 and system 100 for enhanced visualization of a surgical needle 10 in ultrasound data by automatically adjusting ultrasound needle recognition parameters. In accordance with various embodiments of the invention, the method 200 comprises determining 210, by a processor 132, 150 of an ultrasound system 100, a position and orientation of a surgical instrument 10 based at least in part on tracking information emitted by an emitter 14, 112 of a tracking system and detected by a sensor 112, 14 of the tracking system. The sensor 112, 14 and the emitter 14, 112 may be attached to or within a different one of a probe 10 of an ultrasound system 100 and the surgical instrument 10. The method 200 comprises determining 212, by the processor 132, 150, an ultrasound imaging parameter based at least in part on the determined position and orientation of the surgical instrument 10. The method 200 comprises applying the ultrasound imaging parameter to acquire 214, by the probe 104, ultrasound image data of a target. The method 200 comprises generating 216, by the processor 132, an ultrasound image based on the acquired ultrasound image data of the target. The ultrasound image comprises a representation of the surgical instrument 10.

In various embodiments, the surgical instrument 10 is a needle 12. In certain embodiments, the method 200 comprises compounding 216, by the processor 132, 140, the ultrasound image data of the target to generate the ultrasound image. In a representative embodiment, the method 200 comprises performing 202, by the probe 104, an ultrasound scan of patient anatomy to determine that the probe 104 is positioned at the target prior to detecting 210 the tracking information. In various embodiments, the method 200 comprises calibrating 204 the tracking system after the probe 104 is positioned 202 at the target and prior to detecting 210 the tracking information.

In certain embodiments, the emitter 14, 112 is a permanent magnet. In a representative embodiment, the emitter 14, 112 is coupled to the surgical instrument 10. In various embodiments, the tracking information comprises magnetic field strength. In certain embodiments, the tracking system is calibrated with the surgical instrument 10 outside a surgical environment, and comprising introducing 206 the surgical instrument 10 into the surgical environment such that the sensor 112, 14 of the calibrated tracking system detects the magnetic field strength emitted by the permanent magnet 14, 112.

In a representative embodiment, the ultrasound imaging parameter comprises an ultrasound beam steering angle. In certain embodiments, the ultrasound imaging parameter further comprises at least one of a gain, a frequency, a focal zone, a transmit sub-aperture, and a receive sub-aperture. In various embodiments, the representation of the surgical instrument 10 is an image of the surgical instrument 10 when the surgical instrument 10 is in-plane of the ultrasound image data, and a virtual representation of the surgical instrument 10 overlaid on the ultrasound image of the target when the surgical instrument 10 is out-of-plane of the ultrasound image data.

Various embodiments provide a system comprising an ultrasound device 100 that includes a processor 132 and a probe 104. The processor 132, 150 may be operable to determine a position and orientation of a surgical instrument 10 based on tracking information emitted by an emitter 14 of a tracking system and detected by a sensor 112 of the tracking system. The sensor 112 and the emitter 14 may be attached to or within a different one of the probe 104 of the ultrasound device 100 and the surgical instrument 10. The processor 132, 150 can be operable to determine an ultrasound imaging parameter based at least in part on the determined position and orientation of the surgical instrument 10. The processor 132, 150 may be operable to generate an ultrasound image based on ultrasound image data of the target acquired by the probe 104 of the ultrasound device 100. The ultrasound image may comprise a representation of the surgical instrument 10. The probe can be operable to apply the ultrasound imaging parameter to acquire the ultrasound image data of the target.

In a representative embodiment, the ultrasound imaging parameter comprises an ultrasound beam steering angle. In certain embodiments, the ultrasound imaging parameter further comprises at least one of a gain, a frequency, a focal zone, a transmit sub-aperture, and a receive sub-aperture. In various embodiments, the representation of the surgical instrument 10 is an image of the surgical instrument 10 when the surgical instrument 10 is in-plane of the ultrasound image data, and a virtual representation of the surgical instrument 10 overlaid on the ultrasound image of the target when the surgical instrument 10 is out-of-plane of the ultrasound image data. In a representative embodiment, the processor 132, 140 is operable to compound the ultrasound image data of the target to generate the ultrasound image. In certain embodiments, the tracking information comprises magnetic field strength. In various embodiments, the surgical instrument 10 is a needle.

Certain embodiments provide a non-transitory computer readable medium having stored computer program comprises at least one code section that is executable by a machine for causing the machine to perform steps 200 disclosed herein. Exemplary steps 200 may comprise determining 210 a position and orientation of a surgical instrument 10 based on tracking information emitted by an emitter 14 of a tracking system and detected by a sensor 112 of the tracking system. The sensor 112 and the emitter 14 may be attached to or within a different one of a probe 104 of an ultrasound system 100 and the surgical instrument 10. The steps 200 can comprise determining 212 an ultrasound imaging parameter based at least in part on the determined position and orientation of the surgical instrument 10. The steps 200 may comprise applying the ultrasound imaging parameter to acquire 214 ultrasound image data of a target. The steps 200 can comprise generating 216 an ultrasound image based on the acquired ultrasound image data of the target. The ultrasound image may comprise a representation of the surgical instrument 10.

In certain embodiments, the ultrasound imaging parameter comprises an ultrasound beam steering angle. In a representative embodiment, the tracking information comprises magnetic field strength. In various embodiments, the surgical instrument 10 is a needle.

As utilized herein the term “circuitry” refers to physical electronic components (i.e. hardware) and any software and/or firmware (“code”) which may configure the hardware, be executed by the hardware, and or otherwise be associated with the hardware. As used herein, for example, a particular processor and memory may comprise a first “circuit” when executing a first one or more lines of code and may comprise a second “circuit” when executing a second one or more lines of code. As utilized herein, “and/or” means any one or more of the items in the list joined by “and/or”. As an example, “x and/or y” means any element of the three-element set {(x), (y), (x, y)}. As another example, “x, y, and/or z” means any element of the seven-element set {(x), (y), (z), (x, y), (x, z), (y, z), (x, y, z)}. As utilized herein, the term “exemplary” means serving as a non-limiting example, instance, or illustration. As utilized herein, the terms “e.g.,” and “for example” set off lists of one or more non-limiting examples, instances, or illustrations. As utilized herein, circuitry is “operable” to perform a function whenever the circuitry comprises the necessary hardware and code (if any is necessary) to perform the function, regardless of whether performance of the function is disabled, or not enabled, by some user-configurable setting.

Other embodiments of the invention may provide a computer readable device and/or a non-transitory computer readable medium, and/or a machine readable device and/or a non-transitory machine readable medium, having stored thereon, a machine code and/or a computer program having at least one code section executable by a machine and/or a computer, thereby causing the machine and/or computer to perform the steps as described herein for enhanced visualization of a surgical needle in ultrasound data by automatically adjusting ultrasound needle recognition parameters.

Accordingly, the present invention may be realized in hardware, software, or a combination of hardware and software. The present invention may be realized in a centralized fashion in at least one computer system, or in a distributed fashion where different elements are spread across several interconnected computer systems. Any kind of computer system or other apparatus adapted for carrying out the methods described herein is suited. A typical combination of hardware and software may be a general-purpose computer system with a computer program that, when being loaded and executed, controls the computer system such that it carries out the methods described herein.

The present invention may also be embedded in a computer program product, which comprises all the features enabling the implementation of the methods described herein, and which when loaded in a computer system is able to carry out these methods. Computer program in the present context means any expression, in any language, code or notation, of a set of instructions intended to cause a system having an information processing capability to perform a particular function either directly or after either or both of the following: a) conversion to another language, code or notation; b) reproduction in a different material form.

While the present invention has been described with reference to certain embodiments, it will be understood by those skilled in the art that various changes may be made and equivalents may be substituted without departing from the scope of the present invention. In addition, many modifications may be made to adapt a particular situation or material to the teachings of the present invention without departing from its scope. Therefore, it is intended that the present invention not be limited to the particular embodiment disclosed, but that the present invention will include all embodiments falling within the scope of the appended claims.

What is claimed is:

1. A method, comprising:
determining, by a processor of an ultrasound system, a position and orientation of a surgical instrument based at least in part on tracking information emitted by an emitter of a tracking system and detected by a sensor of the tracking system, the sensor and the emitter being attached to or within a different one of a probe of an ultrasound system and the surgical instrument;
determining, by the processor, an ultrasound imaging parameter based at least in part on the determined position and orientation of the surgical instrument;
applying the ultrasound imaging parameter to acquire, by the probe, ultrasound image data of a target; and
generating, by the processor, an ultrasound image based on the acquired ultrasound image data of the target, the ultrasound image comprising a representation of the surgical instrument.

2. The method according to claim 1, wherein the surgical instrument is a needle.

3. The method according to claim 1, comprising compounding, by the processor, the ultrasound image data of the target to generate the ultrasound image.

4. The method according to claim 1, comprising performing, by the probe, an ultrasound scan of patient anatomy to determine that the probe is positioned at the target prior to detecting the tracking information.

5. The method according to claim 4, comprising calibrating the tracking system after the probe is positioned at the target and prior to detecting the tracking information.

6. The method according to claim 5, wherein the emitter is a permanent magnet.

7. The method according to claim 6, wherein the emitter is coupled to the surgical instrument.

8. The method according to claim 7, wherein the tracking information comprises magnetic field strength.

9. The method according to claim 8, wherein the tracking system is calibrated with the surgical instrument outside a surgical environment, and comprising introducing the surgical instrument into the surgical environment such that the sensor of the calibrated tracking system detects the magnetic field strength emitted by the permanent magnet.

10. The method according to claim 1, wherein the ultrasound imaging parameter comprises an ultrasound beam steering angle.

11. The method according to claim 10, wherein the ultrasound imaging parameter further comprises at least one of a gain, a frequency, a focal zone, a transmit sub-aperture, and a receive sub-aperture.

12. The method according to claim 1, wherein the representation of the surgical instrument is:
an image of the surgical instrument when the surgical instrument is in-plane of the ultrasound image data, and
a virtual representation of the surgical instrument overlaid on the ultrasound image of the target when the surgical instrument is out-of-plane of the ultrasound image data.

13. A system, comprising:
an ultrasound device comprising:
a processor operable to:
determine a position and orientation of a surgical instrument based on tracking information emitted by an emitter of a tracking system and detected by a sensor of the tracking system, the sensor and the emitter being attached to or within a different one of a probe of the ultrasound device and the surgical instrument,
determine an ultrasound imaging parameter based at least in part on the determined position and orientation of the surgical instrument, and
generate an ultrasound image based on ultrasound image data of the target acquired by the probe of the ultrasound device, the ultrasound image comprising a representation of the surgical instrument; and
the probe operable to apply the ultrasound imaging parameter to acquire the ultrasound image data of the target.

14. The system according to claim 13, wherein the ultrasound imaging parameter comprises an ultrasound beam steering angle.

15. The system according to claim 14, wherein the ultrasound imaging parameter further comprises at least one of a gain, a frequency, a focal zone, a transmit sub-aperture, and a receive sub-aperture.

16. The system according to claim 13, wherein the representation of the surgical instrument is:
an image of the surgical instrument when the surgical instrument is in-plane of the ultrasound image data, and
a virtual representation of the surgical instrument overlaid on the ultrasound image of the target when the surgical instrument is out-of-plane of the ultrasound image data.

17. The system according to claim 13, wherein the tracking information comprises magnetic field strength.

18. The system according to claim 13, wherein the surgical instrument is a needle.

19. A non-transitory computer readable medium having stored thereon, a computer program having at least one code section, the at least one code section being executable by a machine for causing the machine to perform steps comprising:
determining a position and orientation of a surgical instrument based on tracking information emitted by an emitter of a tracking system and detected by a sensor of the tracking system, the sensor and the emitter being attached to or within a different one of a probe of an ultrasound system and the surgical instrument;
determining an ultrasound imaging parameter based at least in part on the determined position and orientation of the surgical instrument;
applying the ultrasound imaging parameter to acquire ultrasound image data of a target; and
generating an ultrasound image based on the acquired ultrasound image data of the target, the ultrasound image comprising a representation of the surgical instrument.

20. The non-transitory computer readable medium according to claim 19, wherein:
the ultrasound imaging parameter comprises an ultrasound beam steering angle,
the tracking information comprises magnetic field strength, and
the surgical instrument is a needle.
